(19) United States
(12) Patent Application Publication
(10) Pub. No.: US 2019/0082966 A1
(43) Pub. Date: Mar. 21, 2019

(54) OBJECT INFORMATION ACQUIRING APPARATUS AND CONTROL METHOD THEREOF

(71) Applicant: CANON KABUSHIKI KAISHA, Tokyo (JP)

(72) Inventor: Shinji Ohishi, Oyama-shi (JP)

(21) Appl. No.: 16/130,138

(22) Filed: Sep. 13, 2018

(30) Foreign Application Priority Data
Sep. 19, 2017 (JP) 2017-178788

(51) Int. Cl. A61B 5/00; A61B 8/00

(52) U.S. Cl. CPC A61B 5/0095; A61B 8/54; A61B 5/708; A61B 5/4312

(57) ABSTRACT

Provided is an object information acquiring apparatus, having: a holding member configured to hold an object; a liquid tank disposed below the holding member, and configured to store acoustic matching liquid; a probe disposed in the liquid tank, and configured to receive an acoustic wave propagated from the object; a position controlling unit configured to control a relative position of the liquid tank and the holding member; and a driving condition determining unit configured to determine driving conditions of the position controlling unit in accordance with a type of the holding member.

### BACKGROUND OF THE INVENTION

### Field of the Invention

The present invention relates to an object information acquiring apparatus and a control method thereof.

### Description of the Related Art

Research on photoacoustic apparatuses, which acquire information inside an object (living body) by allowing light (e.g. laser) irradiated from a light source to the object to propagate into the object, is actively ongoing especially in medical fields. As one photoacoustic imaging technique, photoacoustic tomography (PAT) is proposed. PAT is a technique in which a pulsed light generated from the light source is emitted to an object, an acoustic wave, which is generated by a bio-tissue absorbing the light propagated and diffused inside the object, is received, and the received acoustic wave is analyzed and processed, so as to visualize the information related to the optical characteristic inside the object, which is a living body. To diagnose the bio-tissue using an ultrasonic wave, a frequency band of several MHz to a little over 10s MHz is used. Inside the living body, the ultrasonic wave decays in the process of the ultrasonic wave being propagated. The ultrasonic wave decays even more so as the frequency becomes higher, and major decay makes it difficult to diagnose a deep region of the living body. Therefore, in an ultrasonic diagnostic apparatus, the object is held by a cup molded by a material having high transmittance of light, such as PET (polyethylene terephthalate). Further, the space between the cup and the ultrasonic probe is filled with an acoustic matching liquid having intrinsic acoustic impedance (a product of sound velocity and density) close to that of the living body, and the ultrasonic wave is acquired in this state (Japanese Patent Application Laid-open No. 2015-109948).
Patent Literature 1: Japanese Patent Application Laid-open No. 2015-109948

### SUMMARY OF THE INVENTION

However, depending on the type of cup which holds the object, waves may be generated in the acoustic matching liquid when the cup and the ultrasonic probe are driven, and bubbles may be generated in the acoustic matching liquid. If bubbles in the acoustic matching liquid enter between the cup and the ultrasonic probe and generate an air layer, the ultrasonic wave is reflected by the interface between the air layer and the acoustic matching liquid because of the difference of the acoustic impedances thereof. As a result, this interferes with the detection of the ultrasonic wave. Besides the photoacoustic imaging apparatus, the same problem occurs to other apparatuses which acquire information inside an object by irradiating the object with an acoustic wave, and receiving the reflected wave of this acoustic wave.

The present invention has been devised in light of the foregoing and it is an object of the present invention to reduce the generation of waves in the acoustic matching liquid.

The present invention provides an object information acquiring apparatus, comprising:

a holding member configured to hold an object;

a liquid tank disposed below the holding member, and configured to store acoustic matching liquid;

a probe disposed in the liquid tank, and configured to receive an acoustic wave propagated from the object;

a position controlling unit configured to control a relative position of the liquid tank and the holding member; and

a driving condition determining unit configured to determine driving conditions of the position controlling unit in accordance with the type of the holding member.

The present invention also provides a method of controlling an object information acquiring apparatus, including a holding member configured to hold an object, a liquid tank disposed below the holding member and configured to store acoustic matching liquid, a probe disposed in the liquid tank and configured to receive an acoustic wave propagated from the object, a position controlling unit, and a driving condition determining unit, the method comprising:

a determining step of determining driving conditions of the position controlling unit in accordance with a type of the holding member by the driving condition determining unit; and

a controlling step of controlling a relative position of the liquid tank and the holding member in accordance with the driving conditions by the position controlling unit.

According to the present invention, the generation of waves in the acoustic matching liquid can be reduced.

Further features of the present invention will become apparent from the following description of exemplary embodiments with reference to the attached drawings.

### BRIEF DESCRIPTION OF THE DRAWINGS

FIG. 1 is a diagram depicting a configuration of a photoacoustic apparatus according to Embodiment 1 of the present invention;

FIGS. 2A and 2B explain a problem of a photoacoustic apparatus;

FIGS. 3A and 3B explain parameters (angle, depth) used for the present invention;

FIGS. 4A and 4B explain a cup identifying unit of the present invention;

FIGS. 5A to 5C explain an angle information acquiring unit of the present invention;

FIGS. 6A to 6C explain a depth information acquiring unit of the present invention;

FIGS. 7A and 7B explain a driving condition determining unit of the present invention;

FIGS. 8A and 8B illustrate scanning patterns of the present invention; and

FIG. 9 is a diagram depicting a configuration of a photoacoustic apparatus according to Embodiment 2 of the present invention.

### DESCRIPTION OF THE EMBODIMENTS

Preferred embodiments of the present invention will be described with reference to the drawings. Dimensions, materials, shapes and relative positions of the components described below can be appropriately changed depending on the configuration and various conditions of the apparatus to which the invention is applied. Therefore, the following description is not intended to limit the scope of the present invention.

The present invention relates to a technique to detect an acoustic wave propagated from an object, and generate and acquire the characteristic information inside the object (object information). This means that the present invention may be regarded as an acoustic apparatus or a control method thereof, or an object information acquiring apparatus or a control method thereof. The present invention may also be regarded as an object information acquiring method or a signal processing method. The present invention may also be regarded as a program which causes an information processing apparatus equipped with such hardware resources as a CPU and memory to execute the method, or a computer readable non-transitory storage medium storing this program.

The object information acquiring apparatus of the present invention includes a photoacoustic apparatus utilizing the photoacoustic effect, in which an acoustic wave generated inside the object by irradiating the object with light (electromagnetic wave) is received, and the characteristic information of the object is acquired as an image data. In this case, the characteristic information refers to information on the characteristic values corresponding to a plurality of positions inside the object respectively, and [these characteristic values] are generated using the signal which originated from the received photoacoustic wave.

The object information acquired by the photoacoustic apparatus refers to a generation source distribution of an acoustic wave which was generated by the light irradiation, an initial sound pressure distribution inside the object, or a light energy absorption density distribution and an absorption coefficient distribution which are derived from the initial sound pressure distribution, and a concentration distribution of a substance constituting a tissue. The concentration distribution of a substance is, for example, an oxygen saturation distribution, a total hemoglobin concentration distribution, and an oxy/deoxyhemoglobin concentration distribution.

The object information acquiring apparatus of the present invention includes an apparatus utilizing an ultrasonic echo technique, which acquires object information as image data by transmitting an ultrasonic wave to an object and receiving a reflected wave (echo wave) reflected inside the object. In the case of the apparatus utilizing the ultrasonic echo technique, the object information to be acquired is information reflecting the differences of the acoustic impedances of the tissue inside the object.

As the characteristic information, which is the object information at a plurality of positions, a two-dimensional or a three-dimensional characteristic distribution may be acquired. The characteristic distribution may be generated as image data, which indicates the characteristic information inside the object. The image data may be generated as the three-dimensional volume data by image reconstruction.

The acoustic wave in the present invention is typically an ultrasonic wave, including an elastic wave called a “sound wave” or an “acoustic wave”. A signal (e.g. electric signal) converted from an acoustic wave by a transducer or the like is called an “acoustic signal” or a “reception signal”. Such phrases as an ultrasonic wave or an acoustic wave in this description, however, is not intended to limit the wavelengths of these elastic waves. An acoustic wave generated due to the photoacoustic effect is called a “photoacoustic wave” or a “light-induced ultrasonic wave”. A signal (e.g. electric signal) which originated from a photoacoustic wave is called a “photoacoustic signal”.

### Embodiment 1

Embodiment 1 will be described in detail with reference to the drawings. As a rule, the same composing elements are denoted with a same reference number, and description thereof is omitted.

General Configuration of Apparatus

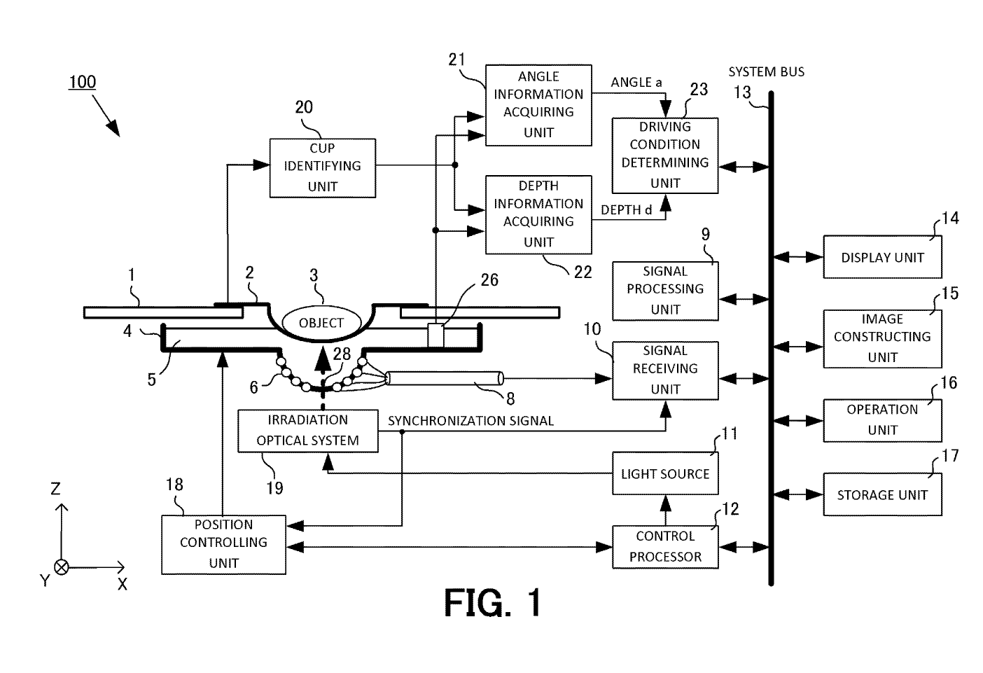

FIG. 1 is a block diagram depicting a photoacoustic apparatus 100 (hereafter called “apparatus 100”) according to Embodiment 1. The apparatus 100 generally has the following composing elements.

A cup 2, which is a holding member, holds an object 3 which is a measurement target. A support unit 1 supports the cup 2. Each of a plurality of ultrasonic probes 6 (probes) receive a photoacoustic wave propagated from the object 3. A position controlling unit 18 controls the relative position of the object 3 and the ultrasonic probe 6. A light source 11 generates light. An irradiation optical system 19 transfer the generated light, and irradiates the object 3 with the light. A photoacoustic signal from the ultrasonic probe 6 is a weak high frequency analog signal, and is transferred to a signal receiving unit 10 via a coaxial cable 8 or the like, so that such an influence as noise from the outside is not received.

The signal receiving unit 10 sends a photoacoustic digital signal, which is generated by amplifying a photoacoustic signal, after converting the photoacoustic signal from analog to digital, to a signal processing unit 9. The signal processing unit 9 performs integration processing and the like on the photoacoustic digital signal to generate the object information. An operation unit 16 receives the input of the instruction information and parameters for the apparatus 100 from the user (e.g. operator who performs inspection, such as medical staff). The instruction information is an imaging start/end instruction, for example, and the parameters are imaging conditions, for example. An image constructing unit 15 generates an image based on the acquired object information. A display unit 14 displays a generated image and the user interface (UI) to operate the apparatus.

A control processor 12 receives various operations from the user via the operation unit 16, generates control information required to generate the target object information, and controls each function via a system bus 13. A storage unit 17 stores the acquired photoacoustic digital signal data, generated image data, and information on other operations. The object 3 to be imaged is a breast, for example, in the case of a breast cancer diagnosis in a Breast Oncology Department, and limbs in the case of vascular diagnosis in a Dermatology Department and Orthopedic Department. The other segments of the living body and a non-living body sample, such as a phantom, may also be a measurement target.

Detailed Configuration

Each composing element of the apparatus 100 will be described in detail. The specific materials, shapes, positions, relative positional relationships and the like described below are merely examples, and the present invention is not limited to the following examples, as long as the functions required for each composing element can be implemented.

Cup

The cup 2 is a holding unit to hold the object 3, and stabilizes the form and position of the object 3 during measurement. This means that a certain rigidity is demanded for the cup 2. The cup 2 is preferably formed of a material having high transmittance, in order to transmit the pulsed light 28 emitted from the irradiation optical system 19 to the object 3. It is preferable that the cup 2 is formed by molding such a material as PET (polyethylene terephthalate), acryl and polymethyl pentene. As described above, various objects 3 may be measured, including a breast, and limbs. Therefore, a plurality of types of cups having different shapes are provided, and a cup having an appropriate shape (size, depth) is selected and used in accordance with the object 3.

Sensor Tub

A sensor tub 4 is a member which is disposed below the cup 2, supports the ultrasonic probe 6, and stores liquid like a liquid tank (tub). The sensor tub 4 includes a hemispherical (bowl-shaped) probe on which the ultrasonic probe 6 is disposed, so as to receive the ultrasonic wave from the object 3 efficiently. It is preferable to use a plurality of ultrasonic probes 6 in order to improve image quality and reduce measurement time. In this case, the plurality of ultrasonic probes 6 may be arranged on the probe one-dimensionally (linear), two-dimensionally (planar) or three-dimensionally (stereoscopic). In the case of a three-dimensional arrangement, a concentric or spiral arrangement is preferable. The ultrasonic probe 6 can be any conversion element that receives an acoustic wave and converts an acoustic wave into an electric signal, and conversion elements utilizing the piezoelectric phenomena, the resonance of light or a change in capacitance may be used.

The sensor tub 4 is a member having the shape of a container for containing acoustic matching liquid 5, to suppress the decay of an acoustic wave by acoustically coupling the object 3 and the ultrasonic probe 6. For the acoustic matching liquid 5, water, oil or the like may be used. The acoustic matching liquid may also be disposed between the cup 2 and the object 3. The sensor tub 4 is one-dimensionally or two-dimensionally scanned by the position controlling unit 18, and the relative position between the cup 2 and the ultrasonic probe 6 is controlled. The ultrasonic probe and the liquid tank may be separate components, without being integrated. The scanning of the sensor tub 4 may also be three-dimensionally scanned, but in this case, it is preferable to fill the acoustic matching liquid 5 between the ultrasonic probe 6 and the cup 2.

Signal Receiving Unit

The signal receiving unit amplifies a photoacoustic signal received by the ultrasonic probe 6, in accordance with the synchronization signal that is inputted from the irradiation optical system 19, and converts the amplified photoacoustic signal into a digital signal, that is, a photoacoustic digital signal. The signal receiving unit 10 is constituted by a signal amplifying unit that amplifies an analog signal from the ultrasonic probe 6, and an A/D converting unit that converts the analog signal into a digital signal.

Signal Processing Unit

For the photoacoustic digital signal generated by the signal receiving unit 10, the signal processing unit 9 corrects the sensitivity dispersion of the ultrasonic probe 6, and performs interpolation to estimate the values of transducers, which are not physically or electrically present. The signal processing unit 9 can also perform integration processing to reduce noise. A photoacoustic signal, that is acquired by detecting the photoacoustic wave emitted from a light absorbing substance inside the object 3, is normally a weak signal. By performing integrating and averaging processing on the photoacoustic signals which were repeatedly acquired from the object at a same position, SN of the photoacoustic signals can be improved with reducing the system noise. The signal receiving unit 10 and the signal processing unit 9 may be constructed by elements (e.g. an A/D converter, a signal amplifier, an adder), and circuits (e.g. FPGA, ASIC).

Light Source

For the light source 11, a solid-state laser (e.g. Yttrium-Aluminum-Garnet laser, Titan-Sapphire laser), which can emit a pulsed light (width: 100 nsec or less) having a central wavelength in the near infrared region, is normally used. Such a laser as a gas laser, dye laser and semiconductor laser may also be used. And instead of laser, a light emitting diode, a flash lamp or the like may also be used.

The wavelength of the light is selected in accordance with the light absorbing substance inside the object to be measured. The light absorbing substance is, for example, oxyhemoglobin, deoxyhemoglobin, blood vessels which contain large quantities of oxy/deoxyhemoglobin, a malignant tumor containing many newly generated blood vessels, glucose and cholesterol. A case of measuring hemoglobin inside a newly generated blood vessels of a breast cancer will be considered, for example. Hemoglobin normally absorbs light in a 600 to 1000 nm range. The light absorption of water constituting a living body, on the other hand, reaches the minimum at around 830 nm, hence light absorption by hemoglobin becomes relatively high in a 750 to 850 nm range. The absorptivity of the light changes depending on the wavelength of the light due to the state of hemoglobin (e.g. the bonding state of hemoglobin and oxygen), therefore the functional change of the living body can be measured using this dependency on wavelength. In the case of measuring the oxygen saturation degree and substance concentration, it is preferable to use a light source which can radiate light beams with a plurality of wavelengths (e.g. a wavelength-variable laser light source, a light source in which a plurality of lasers having mutually different emission wavelengths are combined).

Control Processor

The control processor 12 operates an operating system (OS) which controls and manages basic resources in the program operation, reads the program codes stored in the storage unit 17, and executes the functions described below. The control processor 12 also receives an event notification which is generated in various operations (e.g. imaging start), performed by the user via the operation unit 16, and manages operations to acquire the object information. Further, the control processor 12 controls each hardware via the system bus 13. Furthermore, the control processor 12 controls the irradiation of the pulsed light 28 which is required to generate the target object information, and controls the position of the ultrasonic probe 6 using the position controlling unit 18.

Display Unit

The display unit 14 displays a photoacoustic image constructed by the image constructing unit 15, and the UI to operate images and apparatuses. For the display unit 14, a liquid display, organic EL (Electro Luminescence) display or the like may be used.

Image Constructing Unit

The image constructing unit 15 generates images of the tissue information inside the object based on the photoacoustic digital signal. Then, the image constructing unit 15 constructs an image data so that a 3D display image, a tomographic image at an arbitrary cross-section or the like is displayed on the display unit 14. Further, by applying various correction processing operations such as brightness correction, distortion correction, and extraction of region of interest, to the constructed image, the image constructing unit 15 constructs information that is more relevant for diagnosis. Furthermore, in accordance with the operation by the user via the operation unit 16, the image constructing unit 15 adjusts the parameters related to the configuration of the photoacoustic image, and displays images.

The photoacoustic image is acquired by performing the image reconstruction processing on three-dimensional photoacoustic digital signals generated by the ultrasonic probe 6, and characteristic information (e.g. acoustic impedance) and object information (e.g. optical characteristic value distribution) can be visualized. For the image reconstruction processing, a reverse projection method in the time domain or Fourier domain, a delay and sum method or an inverse problem analysis method using repeated processing, for example, may be used. By using a probe with an acoustic lens or the like having a reception focusing function, the object information may be visualized without performing the image reconstruction.

Operation Unit

The operation unit 16 is an input device for the user to perform image processing operations, such as setting the parameters on imaging (e.g. visualizing range of object information), and instructing the start of imaging. Normally the operation unit 16 is constituted of a mouse, a keyboard, a touch panel and the like, and notifies events for the software (e.g. OS) running on the control processor 12.

Storage Unit

The storage unit 17 is constituted of a memory required for the control processor 12 to operate, a memory that temporarily holds data during the object information acquiring operation, and a storage medium, such as a hard disk, to store generated photoacoustic image data, and related object information and diagnostic information. The storage unit 17 also stores program codes of the software which implements various functions of the apparatus.

Position Controlling Unit

The position controlling unit 18 is a mechanism constituted of mechanical components, such as a motor, an XY stage and a ball screw, for the driving mechanism and driving force transfer mechanism. The position controlling unit 18 controls the position of the ultrasonic probe 6 installed in the sensor tub 4 in accordance with the control information (e.g. acceleration, speed, position) from the control processor 12. By repeating the signal acquisition while emitting the pulsed light 28 to the object 3 and two-dimensionally scanning the ultrasonic probe 6, a wide range of object information can be acquired. The position controlling unit 18 outputs the current position control information to the control processor 12, synchronizing with each emission control of the pulsed light 28 by the irradiation optical system 19.

The above mentioned image constructing unit 15 and the later mentioned cup identifying unit 20 (identifying unit), an angle information acquiring unit 21, a depth information acquiring unit 22, and a driving condition determining unit 23 may be implemented by an information processing apparatus, which includes such arithmetic processing resources as the control processor 12 and the storage unit 17. Each composing element may be implemented by an independent information processing apparatus (e.g. computer, workstation) or processing circuit, or each composing element may be implemented as an independent program module that is executed by a same information processing apparatus or processing circuit. The display unit 14 and the operation unit 16 may be implemented using a display and input device of the information processing apparatus. If a GPU (Graphics Processing Unit) having a high performance arithmetic processing function and graphic display function is used as the image constructing unit 15, the time required for the image reconstructing processing and display image generation can be decreased. A program which causes the information processing apparatus to function as the image constructing unit, the cup identifying unit, the angle information acquiring unit, the depth information acquiring unit and the driving conditions determining unit, and a non-transitory storage medium which records this program and can be read by the information processing apparatus, are included within the scope of the present invention.

Irradiation Optical System

The irradiation optical system 19 guides the pulsed light emitted from the light source 11 toward the object, forms a pulsed light 28 which is appropriate for signal acquisition, and emits this pulsed light 28 from an emitting end. The irradiation optical system 19 is normally comprised of such optical components as a lens, prism, mirror, light diffusing plate and optical waveguide (e.g. optical fiber). In FIG. 1, the emission end is fixed to the base of the hemispherical probe. In the case of this configuration, the light irradiation region on the object and the acoustic wave receiving region by the ultrasonic probe can be linked.

The irradiation optical system 19 detects the emission of the pulsed light 28, and generates a synchronization signal to control the reception and recording of the photoacoustic signal synchronizing with the emission of the pulsed light 28. The emission of the pulsed light 28 can be detected by, for example, dividing a part of the pulsed light generated by the light source 11 using such an optical system as a half mirror, guiding this light to the optical sensor, and the optical sensor generating a detection signal based on the guided light. If a bundle fiber is used to guide the pulsed light, a part of the fibers is branched and guided to the optical sensor, whereby the pulsed light can be detected. The synchronization signal generated based on this detection is inputted to the signal receiving unit 10 and the position controlling unit 18.

Wave Generating State

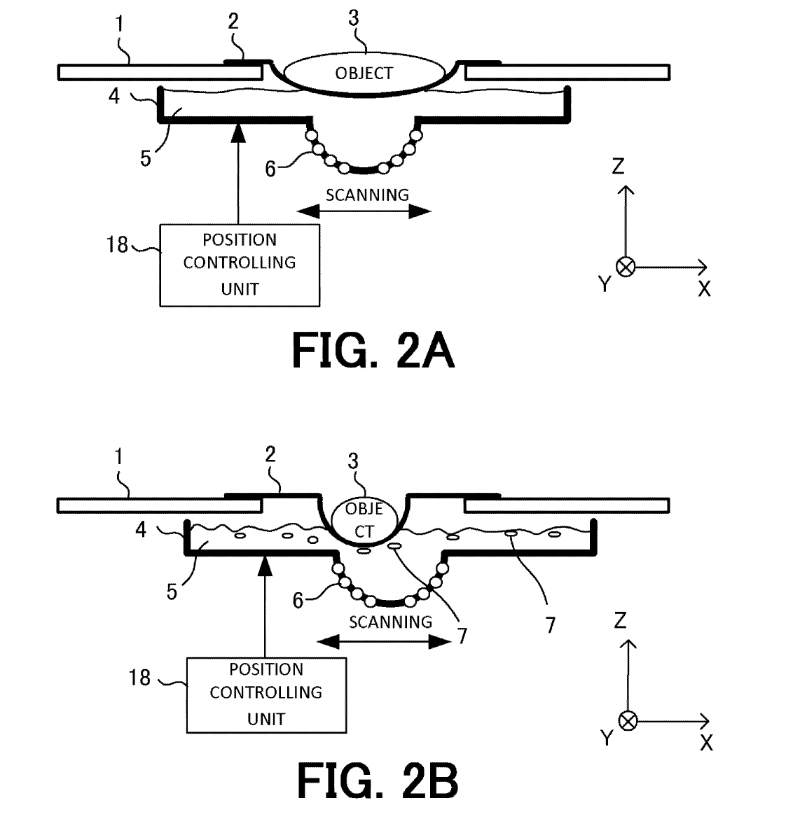

A problem of acquiring a photoacoustic image by scanning the ultrasonic probe 6 will be described with reference to FIGS. 2A and 2B. As described above, various objects may be measured, including a breast and limbs, therefore a plurality of types of cups having different shapes are provided, and the cup having an appropriate shape (size, depth) is selected and used in accordance with the object 3, so that the object 3 does not move during imaging. FIG. 2A is a case when the object 3 is held by the cup 2 having a large diameter and shallow depth, and the image is acquired by scanning the ultrasonic probe 6. In this case, the fluid resistance which the cup 2 receives from the acoustic matching liquid 5 during scanning is small, because the contact angle formed by the end face (side face) of the cup 2 and the liquid surface of the acoustic matching liquid 5 is small. Hence waves are not generated in the acoustic matching liquid 5, even if the scanning speed is relatively fast.

FIG. 2B, on the other hand, is a case when the object 3 is held by the cup 2 having a small diameter and deep depth, and the image is acquired by scanning the ultrasonic probe 6. In this case, the fluid resistance, which the cup 2 receives from the acoustic matching liquid 5 during scanning, is large because the contact angle formed by the end face (side face) of the cup 2 and the liquid level of the acoustic matching liquid 5 is large. Therefore, major waves may be generated in the acoustic matching liquid 5 and bubbles may be generated in the acoustic matching liquid if the scanning speed is fast. Further, bubbles 7 generated in the acoustic matching liquid may enter the space between the cup 2 and the ultrasonic probe 6, and generate an air layer there. If an air layer is generated, the ultrasonic wave is reflected by the interface between the air layer and the acoustic matching liquid because of the difference of acoustic impedances thereof, which interferes with the detection of the ultrasonic wave, and results in difficulty in acquiring a good photoacoustic image.

Characteristic Configuration and Control of Present Invention

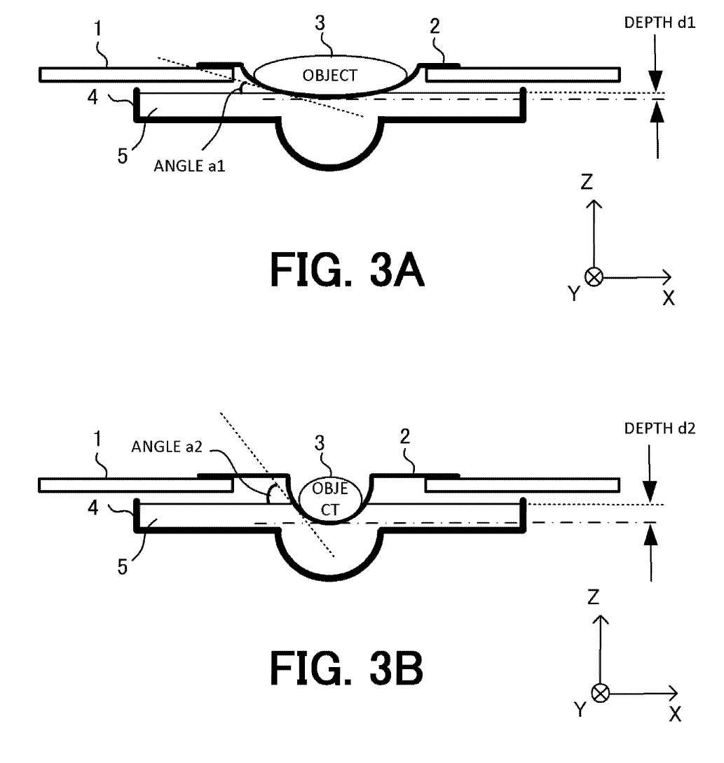

In the present invention, as illustrated in FIGS. 3A and 3B, that angle a formed by the liquid surface of the acoustic matching liquid 5 and the tangential line of the end face (side face) of the cup 2 on the liquid surface (this angle is also called the “contact angle”), and the depth d from the liquid surface to the bottom face of the cup 2 holding the object 3, are used as the parameters to perform control. Even if the cup 2 is replaced with a cup 2 that is optimized for the object 3 and the shape (size, depth) of the cup changes, the generation of waves in the acoustic matching liquid 5 can be suppressed by controlling the scanning conditions in accordance with the parameters (angle a, depth d).

The shape of the holding member of the object 3 may be rectangular or trapezoidal instead of a hemispherical cup shape. Even in the case of using these shapes, the angle formed by the liquid surface of the acoustic matching liquid 5 and the tangential line of the side face of the holding member on the liquid surface is assumed to be the angle a (contact angle). The angle a can be determined, for example, by acquiring the tangential plane of the holding member on the liquid surface, and determining the cross-section when this tangential plane is sectioned by a vertical plane. The depth from the liquid surface of the acoustic matching liquid 5 to the bottom face of the holding member is assumed to be the depth d. The depth d can be determined, for example, by determining the distance between the plane which includes the bottom face (lowest end) of the holding member, and the plane which includes the liquid surface of the acoustic matching liquid, as illustrated in FIGS. 3A and 3B.

As illustrated in FIG. 1, the apparatus of Embodiment 1 includes a cup identifying unit 20 which identifies the cup 2, an angle information acquiring unit 21, a depth information acquiring unit 22, and a driving condition determining unit 23, which determines the driving conditions based on the information on the angle a and the depth d.

Cup Identifying Unit 20

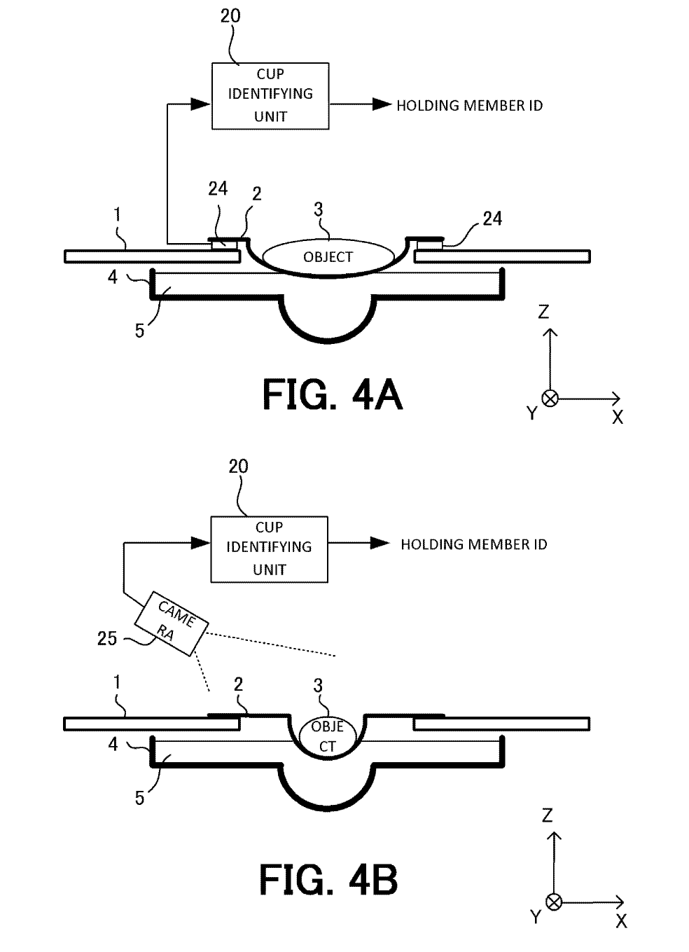

FIGS. 4A and 4B explain the details of the cup identifying unit 20. In FIG. 4A, a holding member identifying member 24 is disposed in the cup 2 to identify the type of the cup. For the holding member identifying member 24, a non-contact type IC tag, constituted of an IC chip for recording data and an antenna for wireless communication, may be used, for example. A holding member ID, to identify the type of the cup, is recorded on the IC chip. The cup identifying unit 20 analyzes the signal from the IC chip, and outputs the holding member ID. The holding member identifying member 24 may have a configuration to identify the type of the chip by a contact type sensor. For example, a correspondence table on the weight and cup type is created in advance, and this correspondence is stored in the storage unit as a reference table. Then a load sensor, such as a load cell, is disposed as the cup identifying unit 20, whereby the control processor 12 can output the holding member ID, referring to the reference table based on the weight of the cup before holding the object.

FIG. 4B illustrates an example when the cup 2 is imaged by the camera 25, then the cup identifying unit 20 performs the image processing to identify the type of the cup 2, and outputs the holding member ID.

Alternatively, a user (e.g. physician, technician) may manually input the size of the holding member and the holding member ID using the operation unit 16 when the cup is replaced. Further, the user may manually input the liquid level, contact angle, depth, driving conditions, correction value and the like via the operation unit.

The holding member identifying member 24 may be a barcode, and the cup identifying unit 20 may be a barcode reader, whereby the type of the cup is identified.

Angle Information Acquiring Unit 21

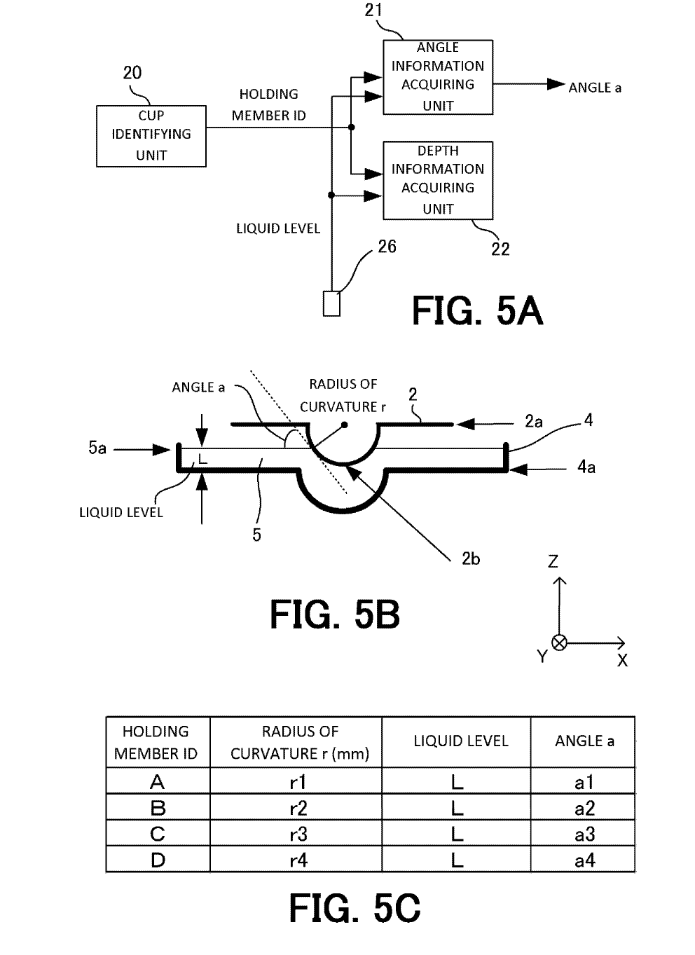

FIGS. 5A to 5C explain details on the angle information acquiring unit. As illustrated in FIG. 5A, the angle information acquiring unit 21 determines the angle a formed by the liquid surface and the tangential line of the cup 2 on the liquid surface based on the holding member ID information from the cup identifying unit 20, and the liquid level information from the liquid level sensor 26.

Here the size of each portion of the cup, as illustrated in FIG. 5B, such as the radius of curvature r of the hemispherical probe and the distance from the surface 2a supported by the support unit 1 to the bottom face (lowest end) 2b, is known. Further, the distance between the bottom face 4a of the sensor tub (bottom face of the liquid tank portion excluding the portion of supporting the ultrasonic probe), and the bottom face 2b of the cup when the cup 2 is installed, is also known based on the design values of the apparatus. If the liquid level L is known by detecting the liquid surface 5a of the acoustic matching liquid 5, the position of the side face of the cup 2 where the liquid surface 5a is located can be calculated. Therefore, as FIG. 5C shows, the angle information acquiring unit 21 can determine the angle a based on the radius of curvature r, which is determined by the liquid level L and the holding member ID. In this example, one liquid level L is used, but the angle a may be calculated in advance for various liquid levels which are determined in accordance with the amount of the acoustic matching liquid 5, and stored in the storage unit 17 in the form of a table or mathematical expression.

Even if the holding member is not cup-shaped, the angle information acquiring unit 21 may acquire the angle a in accordance with the liquid level L using the same method. In other words, the shape of the holding member is known, hence it is easy to store a mathematical expression to calculate the angle in accordance with the liquid level L in the storage unit, or to store the angle a at each liquid level L in the storage unit 17 as a table.

Depth Information Acquiring Unit 22

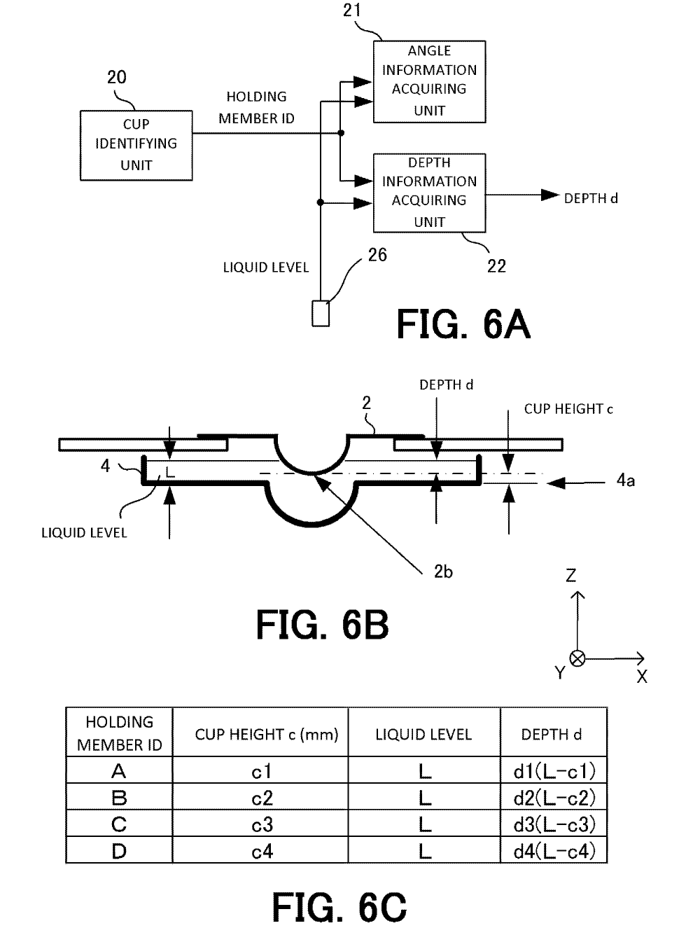

The depth information acquiring unit 22 acquires the depth d, as illustrated in FIG. 6A. In other words, when the cup 2 is installed in the support unit 1, as illustrated in FIG. 6B, the distance from the bottom face 2b of the cup 2 to the bottom face of the sensor tub 4 (cup height c) is uniquely determined based on the design values of the apparatus and the cup. Therefore, a correspondence table between the holding member ID and the cup height c is created and stored in the storage unit 17 in advance. Then if the liquid level L of the acoustic matching liquid 5 is determined by the values detected by the liquid level sensor 26, the depth d from the liquid surface of the acoustic matching liquid 5 to the bottom face of the cup 2, can be acquired.

Driving Condition Determining Unit 23

The driving condition determining unit 23 determines the relative driving conditions of the cup 2 and the ultrasonic probe 6 based on the angle a from the angle information acquiring unit 21 and the depth d from the depth information acquiring unit 22. Here consideration required to set the speed and acceleration in particular will be explained. If the angle formed by the end face of the cup and the acoustic matching liquid surface is small, the fluid resistance, received from the acoustic matching liquid 5, is small, hence few waves are generated in the acoustic matching liquid, even if the scanning speed is relatively fast. Further, if the depth from the acoustic matching liquid surface to the bottom face of the cup is shallow, the fluid resistance, received from the acoustic matching liquid 5, is small, hence few waves are generated even if the scanning speed is relatively fast. On the other hand, if the angle formed by the side face of the cup and the liquid surface is large, or if the depth from the liquid surface to the bottom face of the cup is deep, then fluid resistance is large, and the speed and acceleration must be suppressed.

In the case of FIGS. 3A and 3B, for example, the relationship between the angle a1 of the cup in FIG. 3A and the angle a2 of the cup in FIG. 3B is a2>a1, that is, the scanning speed can be faster in FIG. 3A. The relationship between the depth d1 of the cup in FIG. 3A and the depth d2 of the cup in FIG. 3B is d2>d1, that is, the scanning speed can be faster in FIG. 3A.

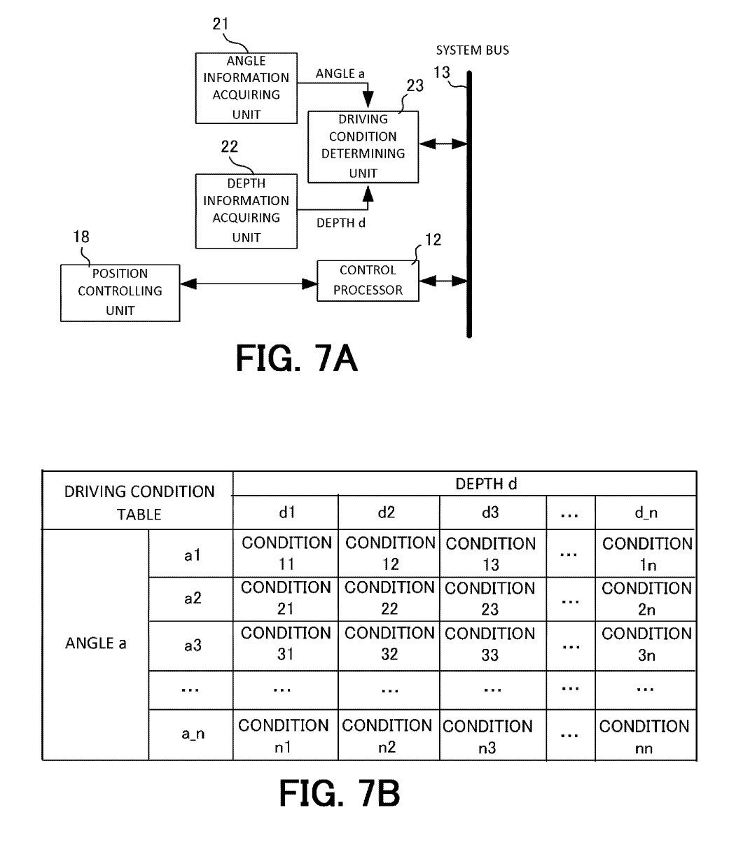

In other words, as the angle between the end face of the cup and the matching liquid surface is sharper (smaller), the relative speed and acceleration between the holding member and the probe can be increased when scanning. Further, as the depth from the matching liquid surface to the bottom face of the cup is shallower (distance thereof is smaller), the relative speed and acceleration between the holding member and the probe can be increased when scanning. Therefore, as depicted in FIGS. 7A and 7B, the behavior (degree of wave generation) of the acoustic matching liquid 5 is observed, and the driving condition table is created in advance using the angle a and the depth d as parameters. The observation can be performed by an observation unit, such as a camera. Even if the cup 2 is replaced with one that is optimized for the object 3, and the shape (size, depth) of the cup changes, the generation of waves in the acoustic matching liquid 5 can be suppressed by determining the driving conditions with reference to the driving condition table. The driving conditions may be created in table format based on a plurality of conditions determined by actual observation, and stored in the storage unit 17. The values between the conditions determined by actual observation may be calculated by interpolation, and created as a mathematical expression.

The driving conditions are sent from the driving condition determining unit 23 to the control processor via the system bus 13, and in the end, the position controlling unit 18, having a motor and the like, performs driving. The driving conditions are, for example, the relative speed and acceleration between the cup and the ultrasonic probe.

Scanning Locus

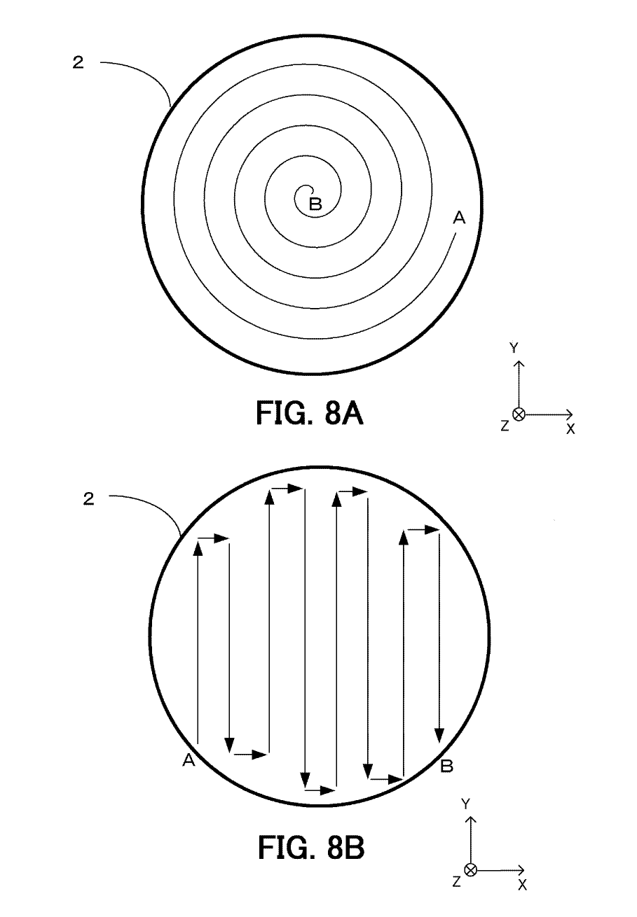

An example of the driving pattern when the ultrasonic probe 6 scans the object 3 will be described. FIG. 8A shows a spiral scanning of which locus is a spiral. FIG. 8B shows raster scanning where the main scanning and sub-scanning are repeated. Both FIG. 8A and FIG. 8B illustrate the loci formed by the center points of the plurality of ultrasonic probes 6 when the cup 2 is viewed from the top (object side), and the start point is indicated by the reference sign A and the end point is indicated by the reference sign B. In both drawings, the start point and the end point are not limited to these points. The rotating direction and the scanning direction are not limited to these either. Whichever is appropriate may be used considering the resolution of the image and the scanning time that are desired. In the case of spiral scanning, the scanning speed largely influences the generation of waves in the acoustic matching liquid at the periphery where peripheral speed is fast. Therefore, the scanning speed may be controlled, depending on the type of cup, at the periphery, but may not be controlled depending on the type of cup at the center portion. In the case of raster scanning, the acceleration changes considerably when the scanning direction changes, hence this must be considered when the driving conditions are set.

As described above, according to the method of Embodiment 1, the driving conditions are controlled by the shape of the holding member, such as a cup, and the level of the liquid surface. Therefore, even if the type of the holding member is changed, the relative driving conditions between the holding member and the ultrasonic probe can be appropriately controlled, and the generation of waves in the acoustic matching liquid can be reduced. As a result, the generation of bubbles in the acoustic matching liquid can be suppressed, therefore image deterioration, caused by the entry of bubbles between the holding member and the ultrasonic probe, can be suppressed.

### Embodiment 2

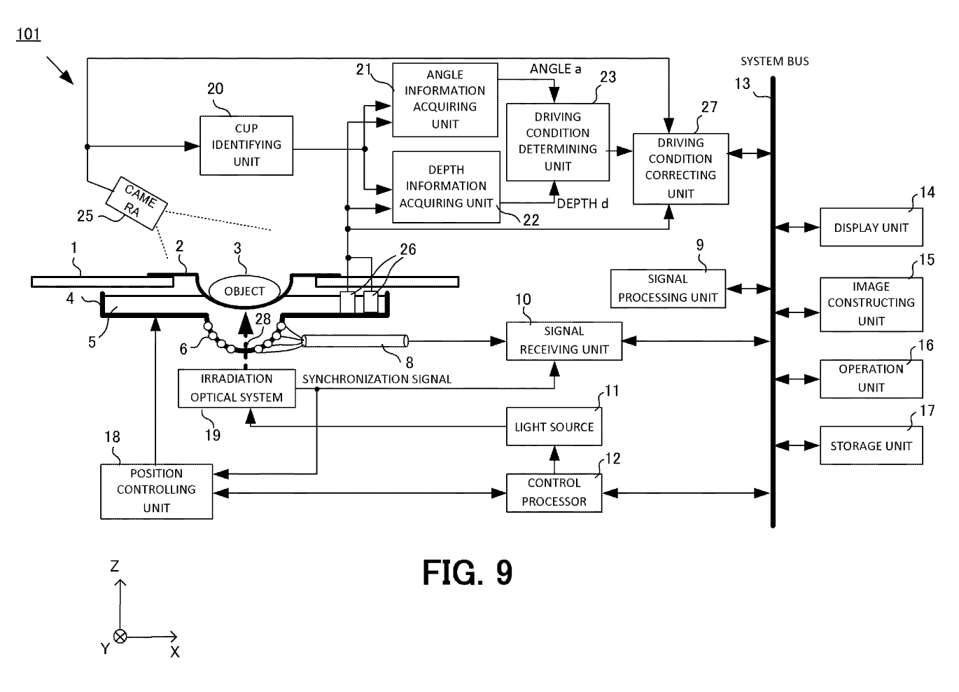

Embodiment 2 will be described in detail with reference to the drawings. FIG. 9 is a block diagram depicting a photoacoustic apparatus 101 (hereafter called “apparatus 101”) according to Embodiment 2 of the present invention. In the apparatus 101, the operation of each portion having the same reference number as the apparatus 100 in Embodiment 1 is the same as the operation of this portion in Embodiment 1. The apparatus 101 in FIG. 9 includes a camera that is used as an observing unit, and a driving condition correcting unit 27. Further, a plurality of liquid level sensors 26 are disposed.

In Embodiment 2 as well, the driving condition determining unit 23 determines the driving conditions based on the angle information from the angle information acquiring unit 21, and the depth information from the depth information acquiring unit 22. In other words, as depicted in FIGS. 7A and 7B, the behavior (degree of wave generation) of the acoustic matching liquid 5 is observed, and the driving condition table is created in advance using the angle a and the depth d as parameters. Thereby even if the cup 2 is replaced with one having a shape (size, depth, angle) in accordance with the object 3, optimum driving conditions can be determined with reference to the driving condition table, and the generation of waves in the acoustic matching liquid 5 can be suppressed.

In a case of interrupting imaging due to an unexpected error or the like in the middle of imaging, and restarting imaging, the behavior (degree of wave generation) of the acoustic matching liquid 5 may become different from previous settings. Here in Embodiment 2, the driving condition correcting unit 27 corrects the driving conditions based on the information from the camera 25, which observes the behavior (degree of wave generation) of the acoustic matching liquid 5, and information on the liquid level from a plurality of liquid level sensors 26. If it is determined that the waves generated in the acoustic matching liquid 5 is greater than the previous setting as a result of processing the information from the camera 25 and information from the plurality of liquid level sensors 26, the driving conditions can be corrected in real-time by decreasing the relative speed, for example.

According to Embodiment 2, in addition to the effects of Embodiment 1, the driving conditions can be corrected to reduce the generation of waves even if scanning is interrupted due to an unexpected problem, thereby a good photoacoustic image can be acquired.

### Embodiment 3

Embodiment 3 will be described. An object information acquiring apparatus of Embodiment 3 is not a photoacoustic apparatus, but an ultrasonic echo apparatus. Therefore, this object information acquiring apparatus does not include such composing elements as a light source and irradiation optical system. Instead the ultrasonic probe 6 transmits the ultrasonic wave to the object 3, and receives the echo wave which is reflected on or inside the object. The image constructing unit 15 of Embodiment 3 generates the characteristic information of the object based on the electric signal converted from the echo wave.

In this ultrasonic echo apparatus as well, a possible problem is the generation of bubbles caused by the generation of waves in the acoustic matching liquid 5 inside the sensor tub 4 during scanning. Therefore, in Embodiment 3 as well, the driving conditions (e.g. speed, acceleration) are set in advance in accordance with the shape and size of the holding member, or the driving conditions are set in real-time. Thereby even if the holding member is replaced, the generation of bubbles that interfere with imaging can be suppressed, and a good photoacoustic can be acquired.

As described above, according to the present invention, the relative driving conditions between the cup and the ultrasonic probe can be appropriately controlled when the acoustic wave is acquired, so that the generation of waves in the acoustic matching liquid is prevented. As a result, the generation of bubbles in the acoustic matching liquid can be reduced, and good object information can be acquired.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-178788, filed on Sep. 19, 2017, which is hereby incorporated by reference herein in its entirety.

What is claimed is:

1. An object information acquiring apparatus, comprising:
a holding member configured to hold an object;
a liquid tank disposed below the holding member, and configured to store acoustic matching liquid;
a probe disposed in the liquid tank, and configured to receive an acoustic wave propagated from the object;
a position controlling unit configured to control a relative position of the liquid tank and the holding member; and
a driving condition determining unit configured to determine driving conditions of the position controlling unit in accordance with the type of the holding member.

2. The object information acquiring apparatus according to claim 1, wherein the driving condition determining unit determines at least one of speed and acceleration when the relative position is controlled.

3. The object information acquiring apparatus according to claim 2, wherein the driving condition determining unit determines the driving conditions in accordance with a liquid level of the acoustic matching liquid and a shape of the holding member.

4. The object information acquiring apparatus according to claim 3, wherein the driving condition determining unit determines the driving conditions so that at least one of the speed and the acceleration increases as a contact angle between the liquid surface of the acoustic matching liquid and the holding member is smaller.

5. The object information acquiring apparatus according to claim 3, wherein the driving condition determining unit determines the driving conditions so that at least one of the speed and the acceleration increases as a depth from the liquid surface of the acoustic matching liquid to the bottom face of the holding member is shallower.

6. The object information acquiring apparatus according to claim 1, further comprising:
an identifying unit configured to identify a type of the holding member; and
a liquid level sensor configured to acquire a liquid level of the acoustic matching liquid.

7. The object information acquiring apparatus according to claim 6, further comprising a storage unit configured to store the driving conditions in accordance with the type of the holding member and the liquid level.

8. The object information acquiring apparatus according to claim 6, wherein the identifying unit performs identification, based on an identifying member disposed in the holding member, an image of the holding member, or information inputted by a user.

9. The object information acquiring apparatus according to claim 8, further comprising:
an observing unit configured to observe behavior of the acoustic matching liquid; and
a driving condition correcting unit configured to correct the driving conditions, based on the behavior.

10. The object information acquiring apparatus according to claim 1, further comprising an irradiation optical system configured to irradiate the object with light from a light source,
wherein the acoustic wave is a photoacoustic wave generated by the irradiation of the light.

11. The object information acquiring apparatus according to claim 1, wherein the acoustic wave is transmitted from the probe and reflected by the object.

12. A method of controlling an object information acquiring apparatus, including a holding member configured to hold an object, a liquid tank disposed below the holding member and configured to store acoustic matching liquid, a probe disposed in the liquid tank and configured to receive an acoustic wave propagated from the object, a position controlling unit, and a driving condition determining unit,
the method comprising:
a determining step of determining driving conditions of the position controlling unit in accordance with a type of the holding member by the driving condition determining unit; and
a controlling step of controlling a relative position of the liquid tank and the holding member in accordance with the driving conditions by the position controlling unit.

13. The method of controlling the object information acquiring apparatus according to claim 12, wherein in the determining step, at least one of speed and acceleration, when the relative position is controlled, is determined.

14. The method of controlling the object information acquiring apparatus according to claim 13, wherein in the determining step, the driving conditions are determined in accordance with a liquid level of the acoustic matching liquid and a shape of the holding member.

15. The method of controlling the object information acquiring apparatus according to claim 14, wherein in the determining step, the driving conditions are determined so that at least one of the speed and the acceleration increases as a contact angle between the liquid surface of the acoustic matching liquid and the holding member is smaller.

16. The method of controlling the object information acquiring apparatus according to claim 14, wherein in the determining step, the driving conditions are determined so that at least one of the speed and the acceleration increases as a depth from the liquid surface of the acoustic matching liquid to the bottom face of the holding member is shallower.

17. The method of controlling the object information acquiring apparatus according to claim 12,
wherein the object information acquiring apparatus further comprises:
an identifying unit configured to identify the type of the holding member;
a liquid level sensor configured to acquire a liquid level of the acoustic matching liquid;
a storage unit configured to store the driving conditions in accordance with the type of the holding member and the liquid level;
an observing unit; and
a driving condition correcting unit,
wherein the method further comprises:
an identifying step of operating the identifying unit to perform identification, based on an identifying member disposed in the holding member, an image of a holding member, or information inputted by a user;
an observing step of observing behavior of the acoustic matching liquid by the observing unit; and
a driving condition correcting step of correcting the driving conditions based on the behavior by the driving condition correcting unit.

18. The method of controlling the object information acquiring apparatus according to claim 12, wherein
the object information acquiring apparatus further comprises an irradiation optical system configured to irradiate the object with light from a light source, and
the acoustic wave is a photoacoustic wave generated by the irradiation of the light.

19. The method of controlling the object information acquiring apparatus according to claim 12, wherein the acoustic wave is a wave that is transmitted from the probe and is reflected by the object.
